(19) United States
(12) Patent Application Publication
(10) Pub. No.: US 2008/0312718 A1
(43) Pub. Date: Dec. 18, 2008

(54) Skin Stimulation Device and a Method and Computer Program Product for Detecting a Skin Stimulation Location

(75) Inventors: Milind Manohar Kulkarni, Sunnyvale, CA (US); Ramzan Nadaf, Bangalore (IN); Sala Lakshmanan, Bangalore (IN); Jagadeesh Chandra Bose Rantham Prabhakara, Bangalore (IN)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V., Eindhoven (NL)

(21) Appl. No.: 12/097,760

(22) PCT Filed: Dec. 7, 2006
(86) PCT No.: PCT/IB06/54661

§ 371 Date: Jun. 17, 2008

(30) Foreign Application Priority Data
Dec. 19, 2005 (EP) 05112385.9

(51) Int. Cl. A61N 1/08

(52) U.S. Cl. 607/59

(57) ABSTRACT

A skin stimulation device is provided with a stimulator (4) for locally stimulating skin at a skin location on a body part, for applying acupressure for example. The device has a storage unit (42) that stores location information specifying the skin location relative to a feature of the body part. The device contains a camera (2) for capturing image data of the skin. A processing circuit (40) processes the captured image data to determine a position of said feature and to identify the skin location based on the location information and the determined position of the feature. The device then preferably indicates whether the stimulator is at a stimulation location and/or automatically starts stimulation.

The invention relates to a skin stimulation device, provided with at least one stimulator for locally stimulating the skin.

The invention also relates to a skin stimulation location detection method.

In the so-called “Pointer Excel II” and “Pointer Plus” needle less stimulation devices are described. These are skin stimulation devices for applying electro-acupressure.

Acupressure is a technique from traditional Chinese medicine, which involves exerting pressure-to-pressure points on the body. Traditionally pressure is exerted with fingers or hands and it is said to alter an internal flow of a vital force or energy called “Qi”. Next to pressure, other forms of skin-stimulation are said to have the same effect and/or carry the same principle as traditional acupressure, such as stimulation by electromagnetic radiation, heat, electricity, vibration, etc.

Acupressure may for example result in strengthening, calming or removing a blockage of the supposed internal flow. Known from “Traditional Chinese Medicine” theory is that the human body has fourteen “imaginary meridians”, or energy cannels, that carry energy throughout the body. These meridians start at the fingertips, connect to the brain and then connect to the organ associated with the specific meridian. A goal of acupressure may be to release blocked energy by stimulating specific locations (acupoints) along the body's fourteen primary meridians. Studies suggest that applying pressure to certain points causes the brain to release more endorphins, small proteins that act as a natural painkiller. Advantages of using acupressure may include relieving pain, balancing the body and maintaining good health, reducing tension, increasing circulation and enabling the body to relax deeply. One of the branches of acupressure is known as reflexology, where the pressure is applied mainly on the sole of the hand and/or foot.

There are numerous books available that explain the theory and techniques of self-acupressure. Acupressure (and reflexology) may be applied by oneself or to another person. Acupressure is said to be particularly effective when the pressure is applied to the correct acupoints. Specialists know how to identify these acupoints. However, non-specialists may find it difficult to apply pressure to the right acupoints. Identifying these points from a handbook may be erroneous and thus not effective.

It would be desirable to provide skin stimulations devices with a facility to ensure that an actuator is activated at recognized acupoints. This presents the problem of providing the device with a facility that identifies actual skin locations that correspond to predetermined locations that are theoretically known to be acupoints for example.

The device described in the aforementioned publication comprises a probe for providing electrical skin stimulation. The probe also provides for detecting the resistance of the skin at the location of the skin to which the probe is directed. When the resistance of the skin is low, a tone, a LED and/or a display will indicate that the probe is pointing at an acupressure point (acupoint). The user may then press the button to apply a predetermined amount electricity to that location.

This known device does not provide for reliable detection of predetermined skin stimulation locations (acupoints). The device may for example indicate a location of low resistance of the skin, which location may not have the desired effect or may not be effective at all. The device may therefore be unsuitable without the aid of a trained specialist.

Among others, an object of the invention is to provide a skin stimulation device that is able to identify specific skin stimulation locations that correspond to predetermined locations, for example to predetermined skin locations that are theoretically known to be acupoints.

Among others, an object of the invention is to be able to indicate when an actuator of the skin stimulation device is positioned to stimulate the skin at a skin stimulation location that corresponds to a predetermined location, for example to a predetermined location that is theoretically known to be an acupoint, or to indicate how the skin stimulation device should be positioned to stimulate the skin at such a skin stimulation location.

A skin stimulation device according to claim 1 is provided. Herein a camera is used to locate skin stimulation locations defined by stored data. In one embodiment a comparison of captured images of the body part with earlier captured images is used to determine whether the device is located so that the stimulator is at a specified location. In one further embodiment the earlier captured images are obtained by capturing an overview image of the body part, locating the stimulation points in the overview image and identifying image parts in the overview image that will be captured by the camera when the device is held closer to the hand with the stimulator at a stimulation location. In another embodiment the earlier captured images are obtained by training: locating the device with the stimulator at a stimulation location determined with the aid of a handbook or an expert and capturing an image from the camera when the device is thus located.

In an embodiment a skin stimulation location detection method is provided, wherein location information is provided specifying the skin location relative to a feature of a body part, an image of the skin is captured with a camera, image data that represents the image is processed with a processor circuit to determine a position of said feature and the skin location is identified based on the location information and the determined position of the feature. This can be used to realize accurate location detection.

### BRIEF DESCRIPTION OF THE DRAWINGS

FIG. 1 shows a schematic top, side and bottom view of an embodiment of a skin stimulation device;

FIG. 2 shows a schematic diagram of an embodiment of a skin stimulation device;

FIG. 3 shows a flow chart of an embodiment of a method of skin stimulation;

FIG. 4 shows a schematic top, side and bottom view of a skin stimulation device; and

FIG. 5 shows a schematic view of a skin stimulation device.

In this description, identical or corresponding parts have identical or corresponding reference numerals. The description shows exemplary embodiments, which serve merely as illustration. The exemplary embodiments shown should not be construed to be limitative in any manner.

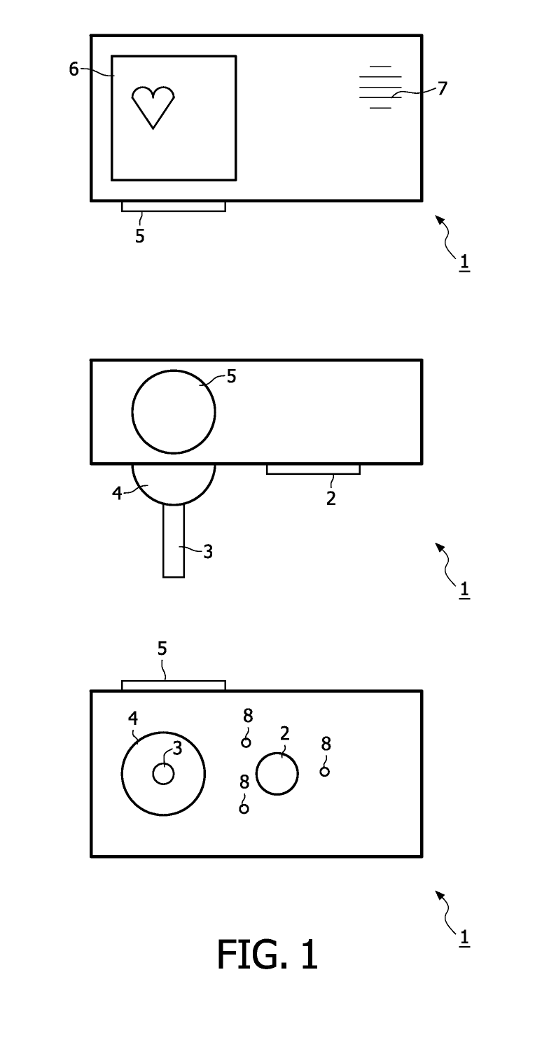

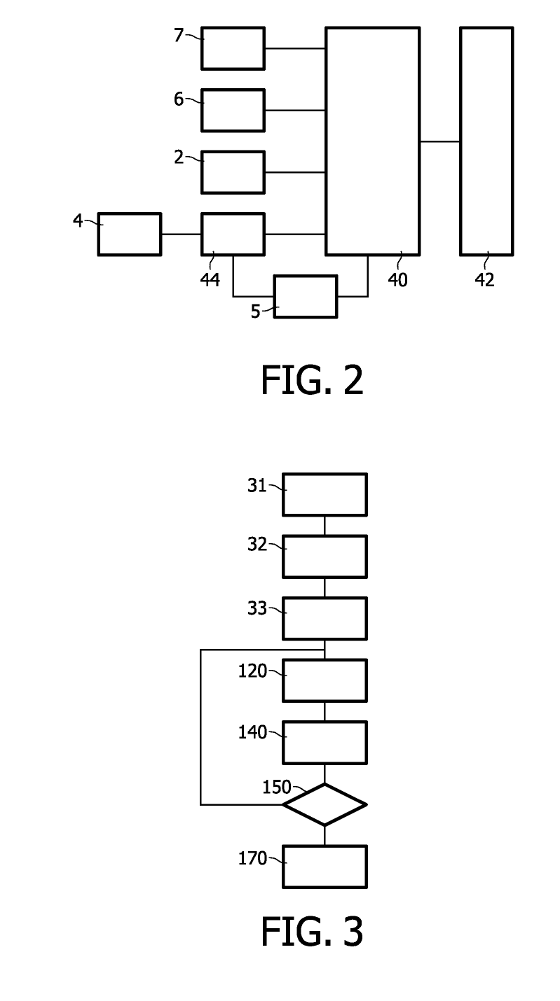

FIGS. 1 and 2 show an exemplary embodiment of an acupressure device 1. FIG. 1 shows the device in top, side and bottom view, respectively. Acupressure device 1 contains a camera 2 to capture an image of the skin of a user, an extendable pointer 3 (shown in extended position), a stimulating knob 4 (which will be more generally referred to as stimulator 4), an actuating button 5, a light source arrangement 8, a display screen 6 and a loudspeaker 7

FIG. 2 shows the functional organization of device 1. In addition to the components the device contains a processing circuit 40, a storage unit 42 and a vibration actuator 44. Actuating button 5 is coupled to vibration actuator 44, to activate vibration actuator 44. Vibration actuator 44 is configured to generate vibrating motions and to transmit these vibrating motions to stimulator 4 for stimulation of the skin of a subject. Storage unit 42 is coupled to processing circuit 40. Processing circuit 40 has an input coupled to camera 2 and outputs coupled to vibration actuator 44, display screen 6 and loudspeaker 7.

Extendable pointer 3 may be telescopic for example, so that it can be pulled out to an extended position and pushed back to a retracted position, wherein it does not extend from device 1. Light source arrangement 8 is configured to illuminate an area at which camera 2 is directed. Processing circuit 40 is configured to receive electronic image signals that represent images captured by camera 2 and to process these electronic image signals.

FIG. 3 shows a flow chart of steps during operation applied to a human hand. Initially pointer 3 is extended and a human user holds the device at a distance to the hand where the extended pointer 3 touches or nearly touches the hand. This distance is selected so that camera 2 is able to capture an image of the entire hand (or a majority part of the hand) and preferably all parts of the hand that contain stimulation locations (for example acupoints, i.e. locations for applying acupressure).

In a first step 31 camera 2 captures an overview image of the hand and stores the captured overview image in storage unit 42. In a second step 32, processing circuit 40 detects one or more visual body features (such as points along outline of a hand or lines on the palm of a hand, a texture pattern etc.) in the image and measures a location or geometric properties of the one or more features in the image. In an embodiment user independent features (i.e. features that are the same for all or most humans) are used at this stage. Methods for detecting the location or properties of visual body features from captured images are known per se and will therefore not be described here.

Storage unit 42 stores location information that defines at least one stimulation location on the hand relative to features of the outline. The stimulation location indicates a location for applying stimulation for a specific desired treatment, for example in fields such as, but not limited to, zone therapy, acupressure, acupuncture, reflexology, massage, etc.

Each location is represented relative to the location of one or more features of the hand. Examples of possible features include fingertips, positions between the points where adjacent fingers extend from the hand, lines from fingertips to these points, lines or crossings of lines in the palm of the hand, etc. In one example, each location is expressed as a set of coordinates in a coordinate system wherein the feature or features has or have known coordinates, orientation and/or size. In another example, each location is expressed in terms of a vector from the location of a feature, the vector being expressed in terms of one or more vectors defined in terms of vectors between locations of features or vectors representing features etc.

In a third step 33 processing circuit 40 uses the detected features and the location information to select locations in the captured image that correspond to the stimulation locations. In one example, offset, rotation and scale factor that relates the coordinates of the features of the hand to the measured location are estimated from the coordinates of a pair of features. In another example the offset, rotation and scale factor are determined that minimize a root mean square error between the measured locations and the locations obtained by applying the offset, rotation and scale factor. In another example a feature that has a certain length and orientation is detected and the offset, rotation and scale factor are estimated from this feature. In yet another example vectors that are used to express the location of stimulation points are estimated from the detected features (which is mathematically equivalent to the estimation of coordinates). Processing circuit 40 determines the stimulation locations in the image from the estimated offset, rotation and scale factor or vectors and the representation of the stimulation locations from storage unit 42.

The relative location of one or more stimulation locations to the detected feature or features is represented by means of parameters that describe how image coordinates in the overview image stored should be translated into the coordinate system wherein the stimulation locations are expressed in the location information, or vice versa, or by stored coordinates of image locations of features with respect to which the stimulation location is defined, or by storing relevant image part or parts for use in matching specifically for the stimulation location or storing the relevant parts specifically for respective stimulation locations, or by storing selection information for extracting these parts from a stored overview image etc.

Subsequently pointer 3 is compressed and the user moves the device closer to the hand, typically to a second distance where stimulator 4 touches the hand. This second distance is so small that camera 2 is able to capture an image of only a part of the hand, near the location where stimulator 4 touches the hand. Processing circuit 40 executes a next step 120 wherein close-up image data is captured by camera 2 and sent to the processing circuit 40. Optionally, processing circuit 40 activates light source arrangement 8 to light the hand during capture of the close up image. In step 140, processing circuit 40 compares the image data with one or more parts of the overview image captured in first step 31. These parts are selected so that these parts correspond to a close-up image (or part thereof) that will be captured when stimulator 4 touches the hand at a location that has been selected in third step 33.

In one embodiment selected image parts are used in said matching, the selected image parts being selected according to the representation made of the stimulation locations relative to the image. Techniques for comparing images or parts thereof are known per se. Preferably, a technique is used that compensates for lighting differences, or removes their effect before matching, so that no exact image content match is needed to detect matching images.

Optionally, a plurality of image parts of the overview image that have no specifically selected relation with a specific stimulation location are matched against the close-up image or a part thereof. In this case, a detected image (part) match is used to determine image coordinates in the overview image of a body part to which camera 2 is directed when it captures the close-up image. These image coordinates are then compared to specified image locations, e.g. by comparing with the computed image coordinates for the image locations, or by converting the image coordinates obtained by matching to model coordinates in a space wherein stimulation locations are specified, and performing the comparison for this space. Incremental searching for matches may be used, wherein processing circuit 40 each time attempts to match with parts of the overview image near a position where a previous match was detected.

If the image data matches stored image data corresponding to skin stimulation locations, this match is detected in step 150. Alternatively when the detected coordinates of camera 2 correspond to those of a stimulation location, this match is detected in step 150. In the flow chart steps 120-140 are repeated for different selected locations if no match is detected. After step 150, when a match is detected, the device 1 will signal the user to activate the stimulator 4 in step 170. In an alternative embodiment processing circuit 40 automatically activates vibration actuator 44 when it is detected that stimulator 4 is at the desired location.

Optionally storage unit 42 (or another storage unit not shown) stores label information linked to the different stimulation locations. In an acupressure application, these labels may identify the type of organ that profits from stimulation of the location for example. In this option processing circuit 40 is arranged to cause the label for a particular stimulation location to be displayed on display screen 6 when it is detected that the stimulator 4 is located at that particular stimulation location from a match between the captured image and the part of the overview image selected for that particular location.

As another option storage unit 42 (or another storage unit not shown) stores duration and/or strength information linked to the different stimulation locations. In an acupressure application, these labels may identify the duration and/or vibration amplitude and/or vibration frequency for each particular location. In this option processing circuit 40 is arranged to read this information from the storage unit 42 for the stimulation location where it has been detected that stimulator 4 is located and to control vibration actuator 44 to apply vibrations with the specified duration and/or vibration amplitude and/or vibration frequency (when the device is activated by hand or automatically). Alternatively or in addition processing circuit 40 causes information derived from these parameters to be indicated on display screen.

Optionally, the device contains a sensor (not shown) to sense a strength of stimulation applied by the user and processing circuit 40 is configured to generate a signal indicating whether the sensed strength is above or below the stored strength (e.g. by displaying both, or displaying a difference on screen 7, or generating a sound signal with a frequency or amplitude dependent on the difference). Similarly, processing circuit 40 may implement a timer function to determine how long a user applies stimulation, the processing circuit generating a signal indicating when the required duration of stimulation has been (or will be) reached.

Preferably, information is stored for a plurality of different points, the information being selected according to the point at which the stimulator 4 is located. In a further embodiment information about a sequence on which the location must be stimulated and optionally a program of stimulation strength and durations in the sequence is stored. In this embodiment processing circuit 40 is arranged to indicate whether the proper point in the sequence is selected and optionally to apply stimulation as defined for the sequence.

It should be appreciated that, although an example has been given for stimulating parts of the hand, similar techniques may be applied for other parts of the body. Similarly, although an example for stimulation by vibration has been given, other forms of stimulation or combinations thereof may be used, such as stimulation by heating, by applying electric current, radiation etc, in which a correspondingly different type of stimulator 4 and driving arrangement will be used instead of stimulating knob 4 and vibration actuator 44.

Although a specific embodiment has been described it should be appreciated that many alternative implementations are possible. For example, although a two stage process has been described wherein first an overview image is captured and later one or more close-ups, it should be appreciated that a one stage process may be contemplated as well. The two-stage process has the advantage that locations can be detected from close-ups when the device is in a position to apply stimulation, even if the close-up distance is too small to provide sufficient image data to locate desirable stimulation locations from information about their position relative to general (person independent) body features.

In an alternative example, the overview image, or parts thereof may be stored for later sessions so that only the matching steps will be needed in those sessions. In this case, the overview image, or parts thereof serves as location information in conjunction with the selected locations during such sessions.

In an alternative embodiment it is not necessary to use an overview image. Matching with a captured image may for example also be advantageous to match the device 1 to specific users, body parts, and/or to (re)set the device 1 with the aid of a specialist and/or handbook. In this case, processing circuit 40 supports switching to a learning mode. When switched to the learning mode processing circuit 40 receives selections from the user, e.g. by detecting when switch 5 is actuated. The user locates stimulator 4 at a stimulation location with the aid of a handbook, or an expert does this for the user, before signaling a selection to processing circuit. In response processing circuit 40 causes camera 2 to capture an image and stores the image for example in storage unit 42, for later use during matching.

This may be repeated for a number of orientations for the same acupoint and/or for a plurality of stimulation locations. Localization of stimulation locations may be more accurate when the data used for matching comprises captured image data for which the user him/herself has indicated the location. This data may be set once or every time before or after stimulation. In this way a flexible and accurate device is obtained.

In this way, the part of the hand (or other body part) from which an image is captured serves directly as a feature for locating stimulation locations.

As another alternative a required overview for locating features of the hand and capturing all stimulation locations by scanning the device over the hand at close up distance, capturing images along the way and obtaining an overview by stitching the captured close-up imaged. In this case, detection whether stimulator 4 is at a stimulation location can start after a sufficient part of the hand has been scanned.

In another alternative embodiment, the close up image capturing stage is eliminated. This is the case for example if stimulation can be provided with device 1 at sufficient distance from the hand to capture an overall image. In this case the locations selected in third step 33 may be used directly to determine whether the stimulator is directed at a desired location.

In an advantageous embodiment, the image processing circuit 40 is configured such that while capturing the image data the user doesn't need to hold the hand at a specific orientation R relative to the device. This may be realized for example by performing the matching step with a plurality of reference images, each corresponding to the expected image when the stimulator is positioned to stimulate a stimulation point when the device is at a respective orientation relative to the hand.

In another embodiment processing circuit 40 is configured to cause a signal to the user to indicate a desired direction of movement towards a skin stimulation location when the stimulator 4 is not positioned at a skin stimulation location. This may be realized for example by matching the captured close up image with parts of the overview image at various locations to detect a current location, and to determine a direction from the current location to a selected desired stimulation location from third step 33 and displaying the direction on display screen 6 and/or giving an audio indication.

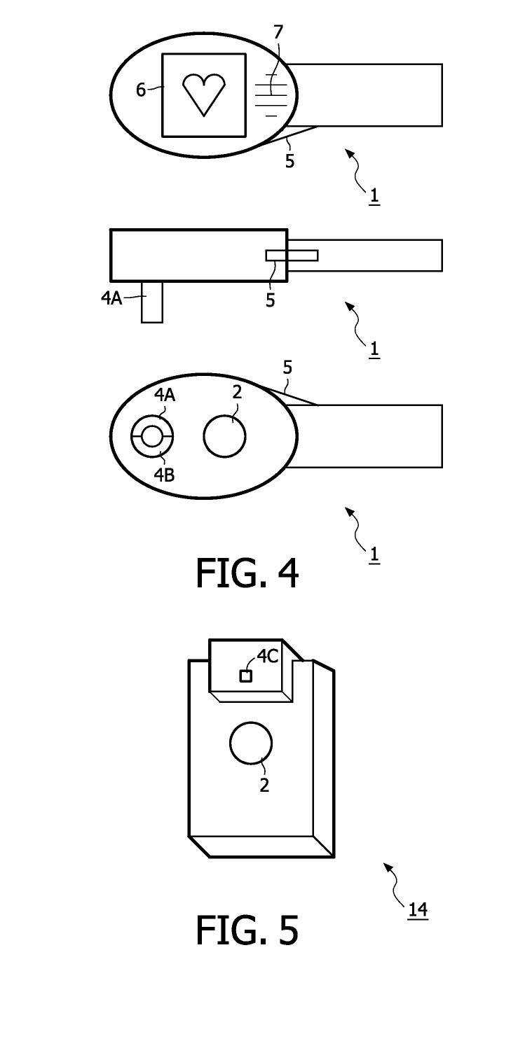

Although an embodiment has been shown wherein the user must start stimulation, in an alternative embodiment processing circuit 40 will itself activate the stimulator 4. In alternative embodiments, the stimulator 4 comprises one, multiple or a combination of electrical, mechanical and/or heating elements such as vibrating elements, infrared lights, electromagnetic radiation elements, electrodes, needles, pressure elements, etc. An exemplary embodiment is shown in FIG. 4, where the stimulator 4 comprises electrodes 4A, 4B to apply stimulation by supplying a current of a pre-defined amount at the acupoints.

Extendable pointer 3 is also optional. Alternatively, the distance may be determined from the size of a detected hand (hand part, or other body part) in the image. In this case processing circuit 40 may be configured to generate a warning signal to change the distance if the distance is not suitable to locate all desired stimulation locations. Optionally, the distance may be measured with a distance sensor (not shown), wherein distance feedback may be signaled to the user with the aid of display 6 and/or speaker 7 so that the user is able to conveniently position the device at a suitable distance for capturing the overview image. Also optionally, a light pointer may be provided and processing circuit 40 may be arranged to direct the light pointer to a stimulation location, that is detected using the overview image, when the device is still at a distance for capturing that overview image. This enables the user to move the stimulator to the indicated location. As an alternative, the device may be equipped with an image projection unit and processing circuit 40 may be configured to project an image onto the hand, wherein one or more stimulation locations are marked. In this case the projected image is offset, scaled and rotated according to the detected position of the hand. This enables the user to move the stimulator to the indicated location. In an embodiment light source arrangement 8 may consist of single light source, or the light source arrangement may be omitted altogether.

Another embodiment is shown in FIG. 5 showing a skin stimulation device 1 according to the invention that can be integrated in a PDA 14 (Personal Digital Assistant) or in a mobile telephone, or implemented in an add-on device for attachment to a PDA or mobile telephone. Thus, this type of device can be made useful to apply treatment to a part of the body. In an embodiment the device 1 comprises an IR heating element 4C for stimulating the skin. Alternatively, any other type of stimulator may be used. Heating element 4C may be connected with the PDA 14 for use of a camera 2 for image capturing. Camera 2 may originally form part of the PDA 14 or mobile telephone. Acupressure software as well as settings may be downloaded to and/or installed with the aid of the processing circuit 40 and storage unit 42 in the PDA 14 so that acupoints may be detected, which will trigger a signal from the PDA 14. Button 5 may be activated to actuate the heating element 4C.

Although a single storage unit 42 has been shown, which main contain a flash memory for example, a more complex storage arrangement may be used, containing for example a RAM memory for captured image data and a ROM for location information etc. Also, an exchangeable memory may be used for stimulation location information, so that different stimulation programs can be plugged in. As another alternative the stimulation program may be downloaded if the device is part of a mobile telephone or a PDA. Any type of memory, or combination thereof may be used, such as a hard disk, a flash memory, battery backed up RAM etc.

Although a preferred embodiment has been shown wherein the camera is located in a fixed relation with respect to stimulator, alternatively a variable relation may be used, the device comprising for example a camera part and a stimulator part, the camera part being used to measure the position of the stimulator with respect to the hand as well, or to indicate a detected stimulation location (e.g. by projected) light. As will be appreciated, this is more complex and less convenient than a device that contains camera and stimulator together in fixed relation.

Processing circuit 40 may be a programmable processor, or a combination of different programmable processors, programmed to perform the described operations and/or processing circuit 40 may comprise dedicated hardware parts, for example for performing matching.

1. A skin stimulation device, provided with
a stimulator (4) for locally stimulating skin at a skin location on a body part;
a storage arrangement (42) for storing location information specifying the skin location relative to a feature of the body part;
a camera (2) for capturing image data of the skin;
a processing circuit (40) configured to process the captured image data to determine a position of said feature and to identify the skin location based on the location information and the determined position of the feature.

2. A skin stimulation device according to claim 1, wherein the processing circuit (40) is configured to determine a position of said feature by matching a stored image, or part thereof, of a part of the body part with an image, or part thereof, captured by the camera (2).

3. A skin stimulation device according to claim 2, wherein the processing circuit (40) is configured to compare a plurality of stored images, of skin parts at respective orientations relative to the skin location with an image, or part thereof, captured by the camera (2) and signaling a detection that the stimulator is located to stimulate the skin location when any of the plurality of stored images matches the captured image or part thereof.

4. A skin stimulation device according to claim 1, wherein the processing circuit (40) is configured to
locate the skin location in the captured image based on the location information and the determined position of the feature;
represent an indication of a part of the captured image that is located in a predetermined spatial relation in the captured image relative to the skin location;
cause storage of at least said part of the captured image data;
to compare said part of the captured image with a further image, or part thereof, from the camera and to identify the skin location based on detection of a match between said part of the captured image and the further image, or part thereof.

5. A skin stimulation device according to claim 4, wherein the processing circuit (40) is configured to represent the relative position by selecting said part of the image in a predetermined spatial relation with respect to the located skin location, the stimulator (4) having a fixed position relative to the camera (2), the predetermined spatial relation being selected so that, when the stimulator (2) is positioned to stimulate the skin location said part of the image is captured by the camera (2).

6. A skin stimulation device according to claim 4, wherein the device comprises an extendible distance indicator (3) for indicating to the user, in an extended position, a distance from the skin for capturing said image data.

7. A skin stimulation device according to claim 1, wherein the camera (2) is located in a fixed spatial relation with respect to the stimulator (4), the processing circuit (40) being configured to generate a human perceptible signal when the processing circuit determines from the location information and the determined position of the feature that the stimulator (4) is positioned to stimulate the skin location.

8. A skin stimulation device according to claim 1, wherein the camera (2) is located in a fixed spatial relation with respect to the stimulator (40), the processing circuit being configured to activate the stimulator (40) in response to determination, from the location information and the determined position of the feature, that the stimulator (40) is positioned to stimulate the skin location.

9. A skin stimulation device according to claim 1, comprising a signal rendering device (6, 7), the storage arrangement (42) being configured to store label information linked to the location information, the processing circuit (40) being configured to cause the rendering device (6, 7) to render the label information when the processing circuit determines from the location information and the determined position of the feature that the stimulator (4) is positioned to stimulate the skin location.

10. A skin stimulation device according to claim 1, comprising a signal rendering device (6, 7), the storage arrangement being configured to store further location information for a further skin location and further label information linked to the further location information, the processing circuit (40) being configured to determine a position of said feature and to identify on which one, if any, of the skin location and the further skin locations the stimulator is positioned to stimulate, and to cause the rendering device to render the label information or the further label information dependent on said determination.

11. A skin stimulation device according to claim 1, wherein said feature is an outline of the body part, the image processing circuit (40) being configured to compare a detected outline of the body part in the captured image data with reference data.

12. A skin stimulation device according to claim 1, comprising a light source arrangement (8) directed at a region to which said camera (2) is directed.

13. A skin stimulation device according to claim 1, wherein the storage arrangement (42) is configured to store stimulation information linked to the location information, representing a degree of stimulation for the skin location.

14. A skin stimulation device according to claim 13, wherein the stimulator (4) is configured to apply a controllable degree of stimulation, the processing circuit (40) being coupled to a control input of the stimulator (4), the processing circuit (40) being configured to control the degree of stimulation dependent on the stimulation information.

15. A skin stimulation device according to claim 14, wherein the degree includes a duration and/or strength of the stimulation.

16. A skin stimulation device according to claim 13, comprising a stimulation indicator and a sensor for measuring a strength of stimulation that is applied by the stimulator, the processing circuit being configured to cause the stimulation indicator to indicate a combination of the stimulation information and a response from said sensor.

17. A skin stimulation device according to claim 1, further configured to operated as a mobile phone or Personal Digital Assistant and/or mobile telephone.

18. A skin stimulation location detection method, the method comprising
providing location information specifying the skin location relative to a feature of a body part;
capturing an image of the skin with a camera (2);
processing image data that represents the image with a processor circuit (40), to determine a position of said feature;
identifying the skin location based on the location information and the determined position of the feature.

19. A method according to claim 18, comprising
locating the skin location in the image based on the determined position of the feature and the location information;
representing an indication of a part of the image that is in a predetermined spatial relation to the located skin location in the image;
storing of at least a part of the captured image data that represents said part of the image;
capturing a further image from a first capture distance from the skin that is smaller than a second capture distance from which the first mentioned image is captured;
comparing said part of the image data with the further image, or part thereof,
identifying the skin location based on detection of a match between said part of the captured image data and the further image, or part thereof.

20. A method according to claim 18 comprising generating a human perceptible signal when it follows from said identifying that a stimulator is positioned to stimulate the skin at the skin location.

21. A method according to claim 19, wherein the location information specifies a plurality of the skin location relative to said feature, the method comprising
providing label information specifying a plurality of labels linked to respective ones of the skin locations;
determining which of the skin locations, if any, the stimulator is positioned to stimulate; and
human perceptibly rendering the label linked to the skin location which the stimulator hast been determined is to be positioned to stimulate.

22. A computer program product comprising a program, which when executed by a programmable computer, causes the programmable computer to perform the steps of the method according to claim 18.
